(19) United States
(12) Patent Application Publication
(10) Pub. No.: US 2024/0198031 A1
(43) Pub. Date: Jun. 20, 2024

(54) MAGNETIC MEANS FOR MOUNTING MASKS ON A WEARER AND MASKS FOR PERFORMING SAME

(71) Applicants: Keng-Ren LIN, Taichung (TW); Matthew LEE, Taipei (TW)

(72) Inventors: Keng-Ren LIN, Taichung (TW); Wan-Ru TSAI, Tainan (TW); Chia-Chi CHIANG, Tainan (TW); Wei-Fang WANG, Tainan (TW); Yueh-Ju LIAO, Tainan (TW); Chien-Fang SU, Tainan (TW)

(21) Appl. No.: 18/555,868

(22) PCT Filed: Apr. 20, 2022
(86) PCT No.: PCT/US22/25445

§ 371 Date: Oct. 18, 2023

Related U.S. Application Data
(60) Provisional application No. 63/285,484, filed on Dec. 3, 2021.
(60) Provisional application No. 63/176,359, filed on Apr. 19, 2021.

(51) Int. Cl. A61M 16/06

(52) U.S. Cl. CPC A61M 16/0688

(57) ABSTRACT

A mechanism for forming a strapless facial mask that operates by magnetically coupling a facial mask to an adhesive coupling adapted to hold and secure the facial mask to the face of a wearer.

### RELATED APPLICATIONS

This application claims benefit to U.S. provisional patent application 63/176,359, filed on Apr. 19, 2021, and U.S. provisional patent application 63/285,484, filed on Dec. 3, 2021. The content of both are incorporated herein by reference in their entirety.

### FIELD OF THE INVENTION

The invention pertains to the field of respiratory facial mask. More particularly, the invention pertains to a means for immobilizing a respiratory facial mask to a wearer's face without the use of a strap. Further, the respiratory facial mask may be fitted to the wearer's face in a customized fit.

### BACKGROUND OF THE INVENTION

Respiratory masks such as oxygen masks for non-invasive ventilation are commonly used in medical settings. However, conventional respiratory masks typically employ facial straps or head straps to hold the mask to a wearer's face. This design, while functional, has several problems.

For example, studies show that patients who need to wear oxygen masks typically will have to wear them for more than 10 days at a time. In such use cases, long term wear of the oxygen mask will cause pressure sore on the patients' faces and ears. This resulted from the fact that the oxygen masks are held against the face by looping the straps over the ears. Straps may irritate the wearer's skin, is uncomfortable to wear and may lead to skin lesion or other injuries. If the mask is applied to a bed-ridden patient who requires the attendance of a caretaker, it is also cumbersome for the caretaker to handle the masks that are physically strapped to the patient via the straps. On a daily-basis, the masks need to be removed and reattached frequently throughout the day for feeding food and drink. In urgent situations, when a patient needs to be intubated, not able to quickly remove the mask may be a serious life-and-death issue. In short, masks utilizing straps are bulky, uncomfortable, unaesthetic, and may result in injuries for long-term wear.

Various attempts in the prior art to address these problem exist. For example, U.S. Pat. No. 6,196,223 to Belfer discloses a strapless respiratory facial mask for attachment to the wearer's face including a moldable laminated gasket member having a cushioning layer and an adhesive layer for engaging the facial contours and skin of the wearer's face. However, Belfer's strapless facial mask still suffers from the problem that once the mask is attached to the face, it is difficult to remove and reattach.

Therefore, there still exists a need for a better mechanism to hold a respiratory facial mask to a wearer's face that does not cause pressure sore and are also easy to be mounted and dismounted frequently.

### SUMMARY OF THE INVENTION

Through trial and error, inventors of the present invention have discovered that a strapless mask may be devised for mounted on the face of a wearer via a magnetic coupling.

Accordingly, in one aspect, the present invention provides a harness for an oxygen mask to be fitted over and securely hold the oxygen mask. Harness in accordance with this aspect of the invention will generally include a main body and one or more connecting elements extending from the main body terminating with a magnetic coupling element for magnetically coupling the harness to a magnetic anchoring element.

In some embodiments, the body of the harness is made of a flexible material. The shape of the body is preferably configured to fit over the oxygen mask. Because there are different sizes of oxygen masks in use today, the body of the harness may be configured to be elastic so that it will fit over different sizes. Alternatively, the size of the harness may be configured to fit over a particular mask.

To securely hold the mask, the body of the harness may be configured as a mesh, preferably an elastic mesh. In some embodiments, the body may be one or more bands instead of a mesh. The bands may further include rigid portions and flexible portions to allow bending.

To provide anchoring force to the mask, the harness will have one or more connecting elements extending therefrom. These connecting elements may be configured as bands or simply a protrusion from the body, or a designated portion of the body configured with a magnetic coupling element. In some embodiments, the harness preferably has two connecting elements extending from each side of the harness's body so that when the body is placed over an oxygen mask, the two connecting elements are positioned on opposite sides of the mask to hold the mask against a wearer's face.

Each of the magnetic coupling elements may be a single magnet or a plurality of magnets. The type of magnet is not particularly limited. Those skilled in the art will be able to adjust the strength of the magnetics to provide suitable anchoring forces.

To hold the mask against a wearer's face, the magnetic coupling elements of the harness are placed in magnetic contact with a matching magnetic anchoring element affixed on the wearer's face. In some embodiments, the magnetic anchoring elements are preferably in the shape of plates, more preferably rectangular plates. Such plates may be configured with magnetic material entirely or just a layer of magnetic material wrapped inside a wrapper. One side of the anchoring element is coated with an adhesive, while the other side provides a contacting surface for the magnetic coupling elements.

In some embodiments, the anchoring element may further be configured with an interlocking element having a magnetic portion configured to couple with an adhesive element which also has a magnetic portion capable of interacting and operatively coupling with the interlocking element via magnetic interaction when the two are brought into close proximity of each other.

In some embodiments, the magnetic coupling element may be configured such that when it is in magnetic contact with the anchoring element, it may be in slidable contact with the anchoring element, i.e. the magnetic coupling element may slide on the anchoring element and still be in magnetic contact. This configuration allows the connecting element a degree of freedom to adjust its anchoring length and angle.

In some embodiments, the anchoring element further includes a protruding edge guard to prevent the magnetic connecting element from sliding off the edge of the anchoring element.

In another aspect, the present invention also provides a strapless mask assembly that can be secured to the face of a wearer. Embodiments in accordance with this aspect of the invention generally include an oxygen mask, a harness as describe above, and a plurality of anchoring element as described above. In some embodiments, the harness and the masks are formed separately. In other embodiments, masks may incorporate the harness as an integrated unit. In such embodiments, there is no distinction of the harness from the mask. The mask itself provides magnetic coupling elements to be coupled with the magnetic anchoring elements.

In yet another aspect, the present invention also provides a method of forming a strapless facial mask. Embodiments in accordance with this aspect of the invention generally includes embedding magnetic coupling elements in the mask. In some embodiments, masks may further include connecting elements extending from the mask terminating with magnetic coupling elements.

Advantageously, the subject technology provides a means for immobilizing a respiratory facial mask on the face of a wearer without the use of a strap. Said means has advantages of affixing the facial mask with relative security such that it does not fall off the wearer's face easily. Masks utilizing said means is also fast to mount onto and dismount from the face of the wearer. It may also be made to accommodate all make and models of masks. Because a soft and air permeable adhesive tape may be used to secure a fit between the mask and the face of a wearer, irritation and injury to the contact skin may also be minimized. Depending on the material used, a face mask utilizing means in accordance with embodiments of the present invention may also be washed and used multiple times.

Other aspects and advantages of the invention will be apparent from the following description and the appended claims.

### BRIEF DESCRIPTION OF THE DRAWINGS

FIG. 1 shows a an arial view of an exemplary facial mask in accordance with embodiments of the present invention.

FIG. 2 shows an exemplary mounting configuration of magnetic adhesive facial strip mounted on a person's face in accordance with embodiments of the presentation.

FIG. 3 shows an exploded view of a strapless facial mask and a wearer in accordance with embodiments of the present invention.

FIG. 4 shows an exploded view of a strapless mask magnetic mounting assembly in accordance with embodiments of the present invention.

FIG. 5 shows an exploded view of a strapless mask mounting assembly in accordance with another aspect of the present invention.

FIG. 6 shows a method of using a strapless mask as a tracheostomy mask.

FIG. 7 shows exemplary embodiments of the harnesses with different shapes of harness body fitting over an oxygen mask. The figure also shows the harness body having extending connecting elements terminating with magnetic elements in magnetic contact with the matching anchoring elements.

FIG. 8 shows a close up picture of an exemplary embodiment of the connecting element extending from the body of the harness and

FIG. 9 shows an exemplary embodiment of the harness body with further bracing elements for improving the fit with the oxygen mask.

FIG. 10 shows an exemplary embodiment of the anchoring element with protruding edge guard.

FIG. 11 shows the exemplary anchoring element of FIG. 10 in use.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced without these specific details. In some instances, well-known structures and components are shown in block diagram form in order to avoid obscuring the concepts of the subject technology.

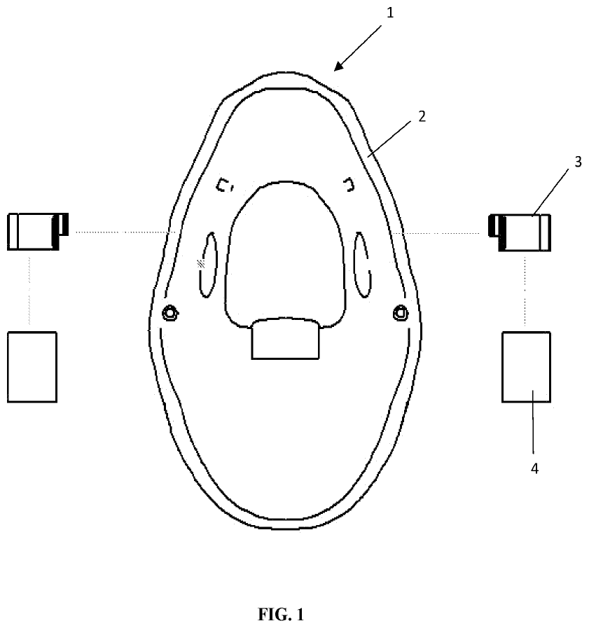

Referring to FIG. 1, there is shown a magnetically mounted strapless facial mask 1 in accordance with embodiments of the present invention. A magnetically mounted strapless facial mask of the present invention will generally have a interfacing cushion 2 disposed on its body, a magnetic mounting mechanism that includes an interlocking element 3 and an adhesive element 4.

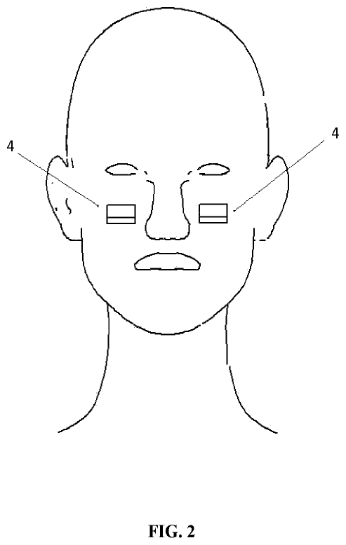

In some embodiments, the adhesive element 4 is disposed on a wearer's face as shown in FIG. 2. The adhesive element 4 is preferably composed of a magnetic material. When affixed to the face of a wearer in a similar manner as illustrated in FIG. 2, the adhesive element 4 may function as a base for receiving and holding a facial mask in place.

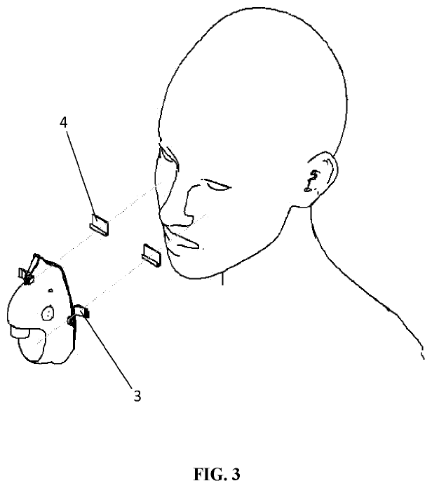

FIG. 3 shows an exploded view illustrating how the facial mask may be mounted to the face of a wearer. As illustrated therein, the exemplary interlock element 3 is configured to function as an anchor for the body of the facial mask. When the facial mask is brought into proximity of the adhesive element 4, the interlocking element 3 form a magnetic bond with the adhesive element 4 and holds the entire facial mask in place without the need for a strap.

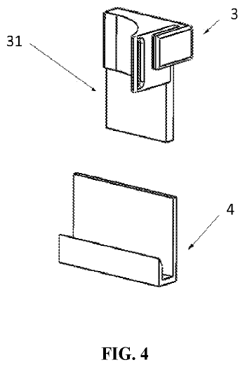

FIG. 4 further illustrates an exemplary embodiment of the interlocking element 3 and the matching adhesive element 4. In this particular embodiment, the interlocking element 3 has a portion configured with magnetic material 31 to interact with the adhesive element 4 which also has magnetic material.

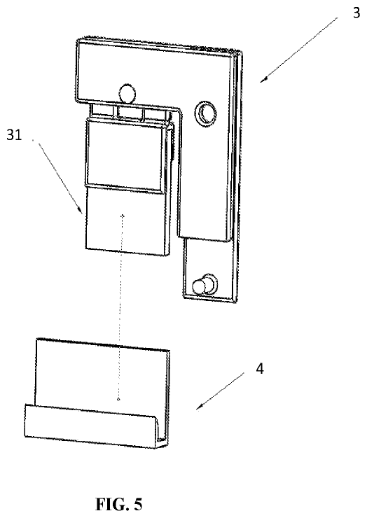

FIG. 5 shows another exemplary embodiment of the interlocking element 3 and the matching adhesive element 4. In this particular embodiment, the interlocking element 3 has a magnetic portion 31 being securely held in place by the body of the interlocking element 3. The magnetic portion 31 is configured to form a magnetic coupling with the adhesive element 4 when brought into proximity of each other.

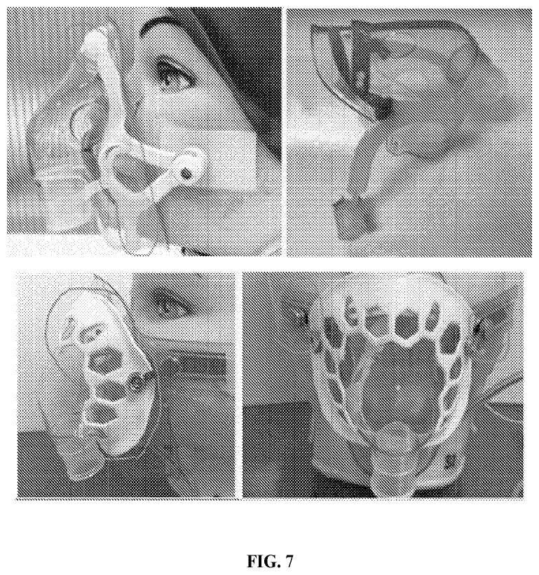

FIG. 7 illustrates various shapes that the harness's body can be configured. Those skilled in the art will recognize that the harness can take any shape so long as it can serve the function of securely holding the oxygen mask. The exemplary embodiments shown in FIG. 7 also illustrates how the harness's body may have extending connecting elements in the shape of two bands terminating with a magnet on each side of the harness to provide anchoring forces.

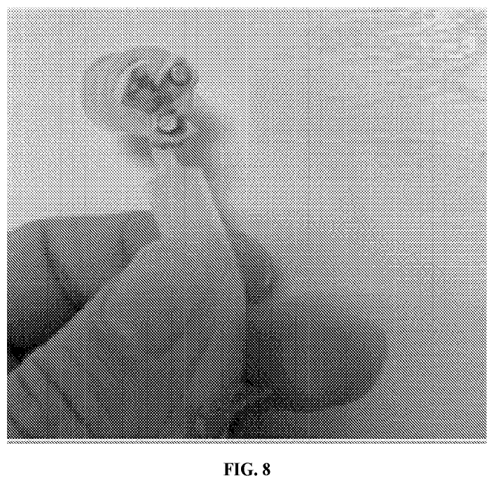

FIG. 8 illustrates an exemplary embodiment of the magnetic coupling element. Here the magnets are formed of three smaller magnets. However, those skilled in the art will recognize that the number, size, and type of magnets are not particularly limited and can be varied to different effects depending on the need.

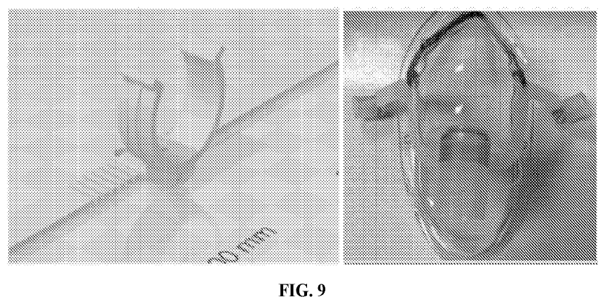

FIG. 9 shows an exemplary embodiment where the body of the harness further includes a bracing element (left panel) which is in the shape matching the air pipe of the oxygen mask. When the harness is placed over the mask, this bracing element will securely lock with the air pipe of the oxygen mask to provide further support. Those skilled in the art will recognized that other additional bracing elements may be introduced to improve “fit” of the harness and the mask, depending on the shape of the mask.

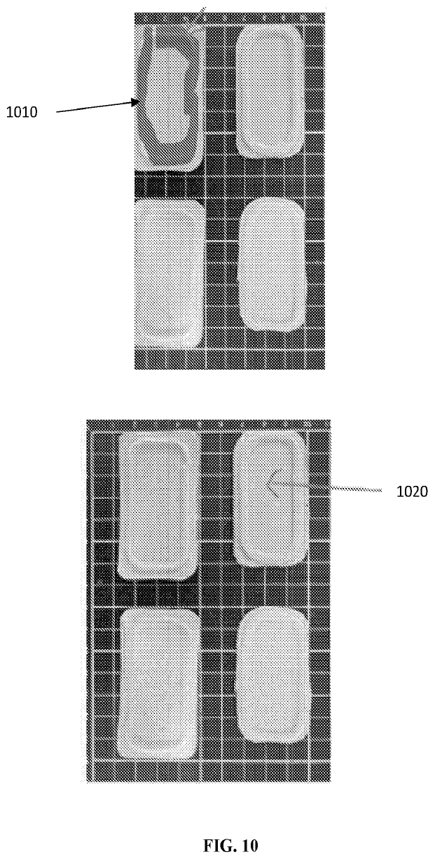

FIG. 10 shows an exemplary embodiment of the anchoring element. As shown in the figure, the anchoring element here is configured as a rectangular plate with an adhesive surface on the back side and the magnetic contacting surface on the front side. Referring to the top panel of FIG. 10, on the front side, the edge of the plate has a protruding edge guard element 1010 to prevent the coupling element of the harness from sliding off the plate. When in use, the coupling magnet may slide with the magnetic contact surface 1020 provided by the anchoring element.

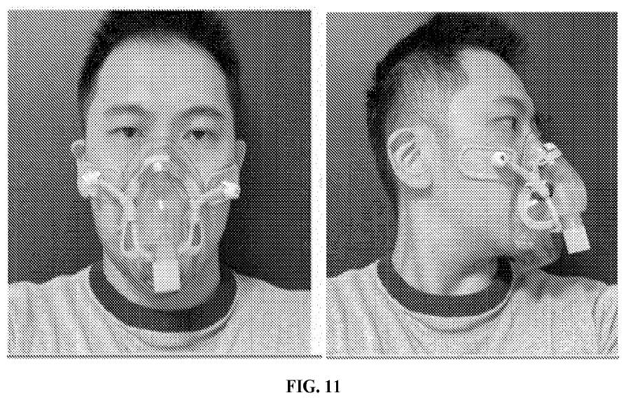

FIG. 11 shows an exemplary use case where the anchoring element is in use. As shown on the right panel of FIG. 11, the harness is heled in place by the connecting elements extending from the harness' body, which in turn holds the mask in place against the user's face. The magnetic coupling element couples the harness-mask assembly to the user's face via its magnetic coupling with the magnetic anchor that is attached to the face of the user. In this configuration, while the mask is held to the user's face, the slidable coupling between the magnetic coupling of the harness and the anchoring element provides certain degree of “lag” so that the pressure asserted on the user's face is minimized, thereby, reducing the chance of pressure sore of the user's face. Further, the magnetic coupling also makes it easy to detach the harness-mask assembly from the user's face and re-mounting it by simply placing the magnetic connecting elements in magnetic contact with the anchoring elements affixed on the user's face.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject technology.

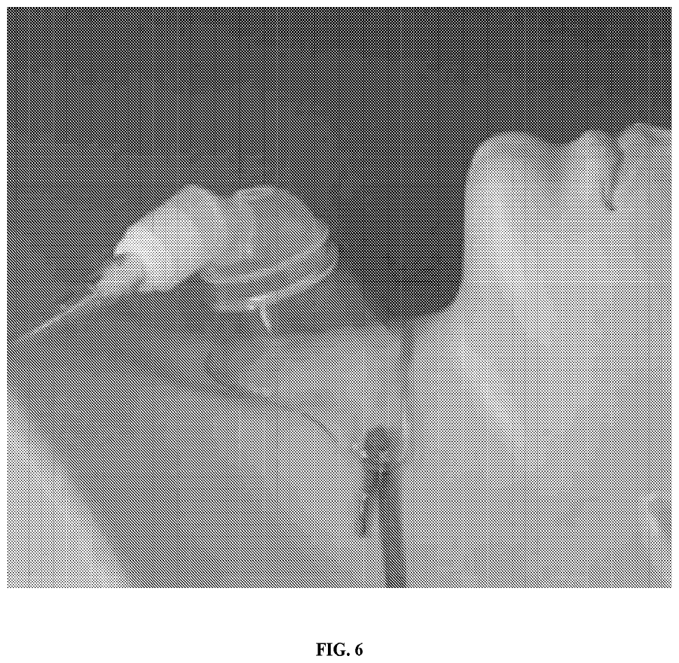

While strapless masks illustrated above are discussed in terms of being used as facial masks, this is not necessarily limited. It will be understood by those skilled in the art that strapless masks will have a wide range of applications other than facial masks. In one exemplary embodiment illustrated in FIG. 6, strapless masks may be configured to function as tracheostomy masks to be placed on a patient's tracheostomy for delivering oxygen or aerosols.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A harness for an oxygen mask, comprising:
a flexible body configured to fit over an oxygen mask; and
one or more connecting portions extending from the body and terminating with a magnetic coupling element for coupling to a corresponding magnetic anchoring element.

2. The harness of claim 1, wherein the body is comprised of a flexible mesh configured to elastically fit over different sizes of oxygen mask.

3. The harness of claim 1, where the body has a first connection portion extending from one end and a second connection portion extending in the opposite direction, each terminating with a magnetic coupling element.

4. A strapless mask assembly, comprising:
a harness having a body and one or more connecting elements extending from the body and terminating with a magnetic coupling element;
a oxygen mask; and
a magnetic anchoring element having an adhesive side and a non-adhesive side,
wherein said anchoring element is configured to be affixed to a wear's face on the adhesive side, and providing a magnetic connection point to the magnetic coupling element of the harness such that when the magnetic coupling element is in magnetic contact with the anchoring element, the harness is anchored to the face of the wearer, thereby, securing

5. A magnetic anchoring element for anchoring the strapless mask assembly of claim 4, comprising:
an adhesive side configured with a suitable adhesive to affix the anchoring element to the wearer's face; and
a non-adhesive side configured to provide a contacting surface for the magnetic coupling element of the harness.

6. The magnetic anchoring element of claim 4, wherein said non-adhesive side

7. A method for detachably mounting an oxygen mask to a wear's face, comprising:
placing the strapless mask assembly of claim 4 in magnetic contact with magnetic anchoring elements of claim 5 affixed on the face of the wearer.

8. A method for detachably mounting an oxygen mask to a wear's face, comprising: agnetic anchor for anchoring the strapless mask assembly of claim 4, comprising:

9. The harness of claim 1, where the body has a first connection portion extending from one end and a second connection portion extending in the opposite direction, each terminating with a magnetic coupling element.
